(19) United States
(12) Patent Application Publication
(10) Pub. No.: US 2017/0028155 A1
(43) Pub. Date: Feb. 2, 2017

(54) COUPLINGS, TRACHEOSTOMY TUBES AND AIRWAY SYSTEMS

(71) Applicant: SMITHS MEDICAL INTERNATIONAL LIMITED, Kent (GB)

(72) Inventors: Timothy Bateman, Kent (GB); Stephen James Field, Kent (GB); Mark Andrew Graham, Kent (GB); Andrew Thomas Jeffrey, Kent (GB)

(73) Assignee: SMITHS MEDICAL INTERNATIONAL LIMITED, Kent (GB)

(21) Appl. No.: 15/303,750

(22) PCT Filed: Mar. 18, 2015
(86) PCT No.: PCT/GB2015/000094

§ 371 Date: Oct. 13, 2016

(30) Foreign Application Priority Data
Apr. 25, 2014 (GB) 1407318.3

(51) Int. Cl. A61M 16/08; A61M 16/04

(52) U.S. Cl. CPC A61M 16/0825; A61M 16/0465; A61M 16/0497

(57) ABSTRACT

A coupling (2) for a tracheostomy tube (1) has first component (21) with a short sleeve (23) at its patient end that it fitted on the patient end of the machine end coupling (17) on the tube so that the machine end of the coupling extends within a part-spherical formation (24) at the opposite end of the first component. The coupling (2) includes a second component (22) with a male tapered fitting (27) at its machine end and a part-spherical formation (28) at its patient end that is a close sliding fit inside the part-spherical formation (24) on the first component (21).

This invention relates to couplings of the kind adapted for fitting on a male connector at the machine end of a tracheostomy tube, the coupling including a first component including a substantially cylindrical sleeve at one end having a female tapered bore adapted to form a tapered fit on the outside of the male connector.

Tracheostomy tubes are used to enable ventilation or respiration of a patient. The tube is inserted into the trachea via a surgically-formed opening in the neck so that one end locates in the trachea and the other end locates outside the patient adjacent the neck surface. Various types of different tracheostomy tubes are presently available to suit different needs. Tracheostomy tubes are also available with an inner cannula, which can be removed and replaced periodically to prevent the build-up of secretions and avoid the need to replace the tube itself. Tracheostomy tubes can be inserted by different techniques, such as the surgical cut down procedure carried out in an operating theatre or a percutaneous dilatation procedure, which may carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. Current medical practice is to encourage tracheostomy patients, where possible, to start moving and become mobile as soon as possible after the tracheostomy procedure. This promotes blood flow and helps to speed the recovery process. If, however, the patient is being ventilated and is connected to a breathing circuit via tubing connected to the 15 mm coupling on the tracheostomy tube, the rigid nature of the connection makes it difficult for the patient to be ambulatory. If the tracheostomy tube is moved relative to the patient by forces applied by the interconnection this can result in pressure, trauma and stenosis to the trachea. It has been proposed to couple the breathing circuit to the tracheostomy tube by means of a ball and socket swivel provided by a ball member at the machine end of the tracheostomy tube and a socket member that is attachable to the breathing circuit. Examples of these arrangements are described in GB2007789 and U.S. Pat. No. 5,259,376. The problem with such arrangements is that they tend not to be very compact and may be difficult to use with conventional couplings and fittings such as inner cannulae.

It is an object of the present invention to provide an alternative coupling, tracheostomy tube assembly and airway system.

According to one aspect of the present invention there is provided a coupling of the above-specified kind, characterised in that the first component has at its opposite end an enlarged part-spherical formation having an open end aligned with the sleeve, that the coupling includes a second component having a machine end portion of substantially cylindrical shape with a male tapered external surface adapted to fit with a cooperating female tapered connector, that the patient end of the second component has a part-spherical formation with an open end, and that the part-spherical formation on the second component is located within the part-spherical formation on the first component and forms a close sliding fit between the outside of the formation on the second component and the inside of the formation on the first component such that the machine end of the second component can be displaced in two orthogonal planes relative to the first component.

The sleeve on the first component is preferably shorter than the male connector on the tracheostomy tube, the sleeve being arranged to fit towards the patient end of the male connector such that the machine end of the male connector extends into the part-spherical formation at the machine end of the first component. The sleeve on the first component is preferably arranged such that about half the length of the male connector extends in the part-spherical formation on the first component. The coupling is preferably arranged such that an end of the male connector projects in the open end of the part-spherical formation on the second component.

According to another aspect of the present invention there is provided a tracheostomy tube assembly including a tracheostomy tube and a coupling fitted on a male connector at the machine end of the tracheostomy tube the coupling including a first component including a substantially cylindrical sleeve at one end having a female tapered bore fitted on the outside of the male connector, characterised in that the first component has at its opposite end an enlarged part-spherical formation having an open end aligned with the sleeve, that the coupling includes a second component having a machine end portion of substantially cylindrical shape with a male tapered external surface adapted to fit with a cooperating female tapered connector, that the patient end of the second component has a part spherical formation with an open end, and that the part-spherical formation on the second component is located within the part-spherical formation on the first component and forms a close sliding fit between the outside of the formation on the second component and the inside of the formation on the first component such that the machine end of the second component can be displaced in two orthogonal planes relative to the first component.

The sleeve on the first component is preferably shorter than the male connector on the tracheostomy tube, the sleeve being fitted towards the patient end of the male connector such that the machine end of the male connector extends into the part-spherical formation at the machine end of the first component. The sleeve on the first component is preferably arranged such that about half the length of the male connector extends in the put-spherical formation on the first component. An end of the male connector preferably projects in the open end of the part-spherical formation on the second component.

According to a further aspect of the present invention there is provided an airway system including a breathing circuit and a tracheostomy tube assembly according to the above other aspect of the present invention, characterised in that the tracheostomy tube is connected with the breathing circuit via the coupling such that movement of the breathing circuit relative to the tracheostomy tube is accommodated by movement of the second component relative to the first component.

A coupling, tracheostomy tube and an airway system according to the present invention will now be described, by way of example, with reference to the accompanying drawings, in which:

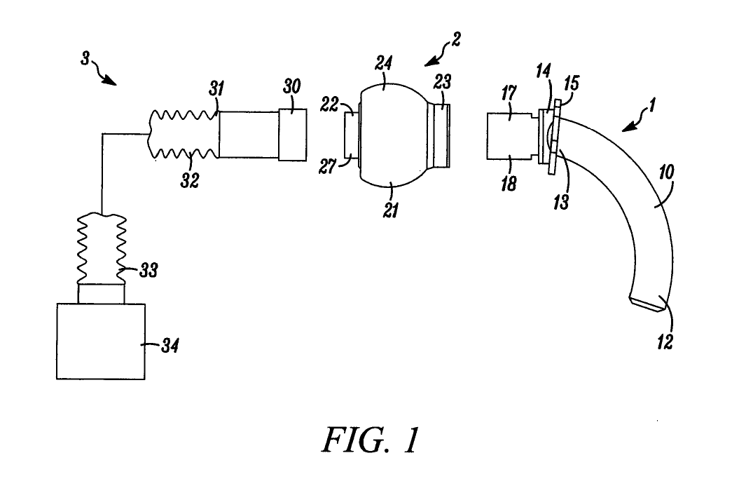

FIG. 1 is a side elevation view showing an airway system including a tracheostomy tube, coupling and breathing circuit separated before connection;

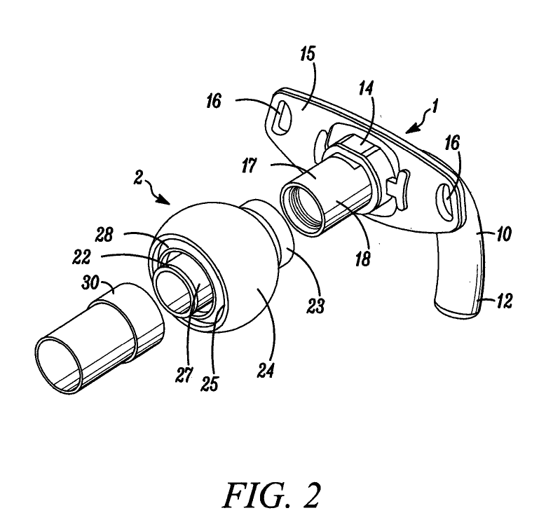

FIG. 2 is a perspective view of a part of the airway system;

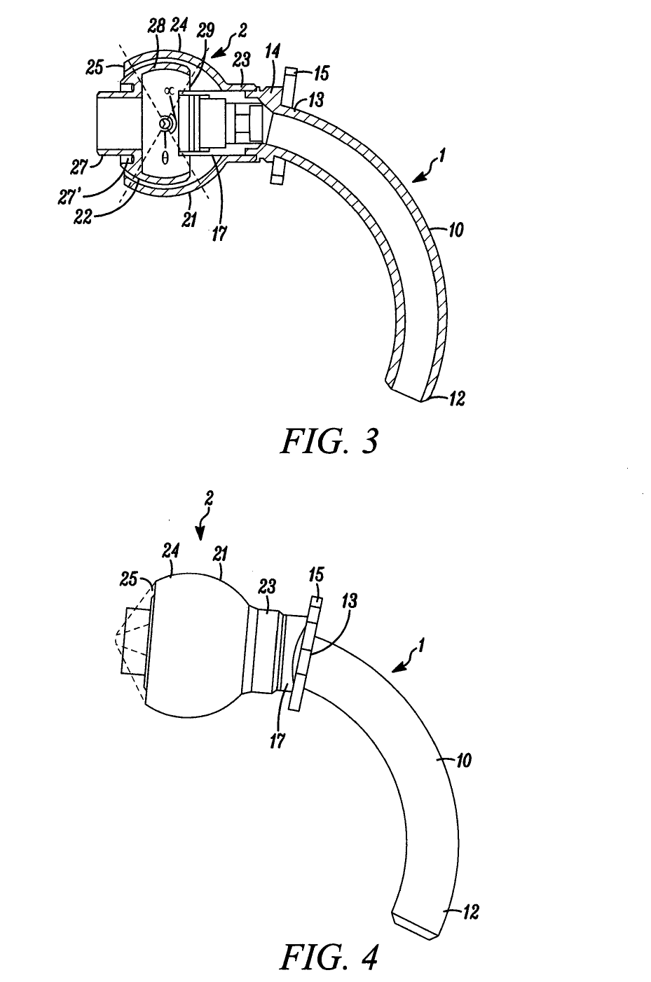

FIG. 3 is a cross-sectional side elevation view of the tracheostomy tube and coupling fitted together; and

FIG. 4 is a side elevation view of the tracheostomy tube and coupling illustrating the range of possible swivelling deflection.

With reference first to FIGS. 1 and 2, the airway system comprises a conventional tracheostomy tube 1, a removable swivel coupling 2 and a breathing circuit 3 that is, in use, connected with the tracheostomy tube via the coupling.

The tracheostomy tube 1 has a curved shaft 10 of circular section with a patient end 12 adapted to be located within the trachea of the patient. The shaft 10 may have a conventional sealing cuff towards its patient end although the tube shown does not have any sealing cuff The shaft 10 is moulded or extruded and is bendable but relatively stiff, being made of a plastics material such as PVC or silicone. The machine end 13 of the shaft 10 is adapted, during use to be located externally and adjacent the tracheostomy opening formed in the patient's neck. The machine end 13 of the shaft 10 supports a relatively stiff hub 14 including a radially-extending support flange 15 that is adapted to lie against the skin surface on either side of the tracheostomy opening. The flange 15 has openings 16 at opposite ends for attachment to a neck strap (not shown) used to support the tube with the patient's neck. A 15 mm male tapered connector 17 is fixed with or forms a part of the hub 14 and has a tapered external surface 18. The connector 17 is adapted to make a removable push fit in the forward, patient end of the swivel, ball and socket coupling 2.

With reference now also to FIGS. 3 and 4, the swivel coupling 2 is formed of a first, patient end, socket component 21 and a second, machine end, ball component 22. The patient end component 21 is moulded of a stiff plastics material and is of hollow construction having a short, substantially cylindrical sleeve 23 at its patient end with an internal, female tapered surface shaped to make a mating slip fit with the mating male connector 17 on the tracheostomy tube 1. The sleeve 23 is about half the length of the male connector 17 and its internal diameter is such that it makes a sliding fit on the forward, patient end of the male connector. The opposite, rear or machine end of the component 21 is formed into a part-spherical formation 24 with a part-spherical shape on both its external and internal surfaces. The external diameter of the part-spherical formation 24 is about twice the internal diameter of the sleeve 23 and the formation extends around an angle θ of about 255° including the sleeve 23. Because the sleeve 23 is relatively short and fits on the forward end of the tube connector 17, the rear end of the connector extends into the part-spherical formation 14 along about half of its length. The rear or machine end of the formation 24 has a circular opening 25 oriented at right angles to the axis of the component 21. The diameter of the opening 25 is about two thirds the external diameter of the formation 24.

The second, machine end component 22 is also moulded from a stiff plastics material and is of a hollow construction having a short sleeve 27 at its rear, machine end that is formed with a taper on its external surface adapted to be received in mating engagement in a standard female tapered 15 mm connector 30. The opposite, forward or patient end of the component 22 is formed with a part-spherical, ball formation 28 having an external diameter that is a close sliding fit inside the part-spherical, socket formation 24 on the first component 21. The ball formation 28 has an opening 29 at its forward, patient end that subtends an angle α of about 125° at the centre of the formation. The ball formation 28 is shorter than the socket formation 24 on the first component, being about two thirds its length. The sleeve 27 extends a short distance within the part-spherical formation 28 forming an external annular recess 27′ around the forward end of the sleeve. The machine or rear end of the sleeve 27 projects from the opening 25 of the outer socket component 21 so that it is accessible by a mating connector 30. The plastics used in the inner and outer components 21 and 22 are selected to allow a close sliding fit to minimise gas leakage between them whilst also minimising friction between them and preventing binding.

The breathing circuit 3 (FIG. 1) includes a standard 15 mm female tapered connector 30 mounted at the patient end 31 of flexible, corrugated gas tubing 32. The machine end 33 of the tubing 32 is connected to a ventilator or anaesthetic apparatus 34. Instead of single limb tubing the circuit could include two parallel tubes for inspiratory and expiratory gas respectively.

The female connector 30 is coupled to the swivel coupling 2 by being pushed on to the outside of the sleeve 27 of the ball component 22. The recess 27′ around the sleeve 27 enables the connector 30 to extend a small distance inside the part-spherical formations 24 and 28 of the swivel coupling 17 thereby to form a secure attachment without the need for the sleeve to project as far out from the coupling 2 as would otherwise be the case.

By arranging for the connector 17 on the tracheostomy tube 1 to extend within the part-spherical formation 14 the overall length of the swivel coupling 2 that projects from the tracheostomy tube can be minimised. This reduces the torque forces on the tracheostomy tube 1, and hence the pressure applied to the trachea, caused by the weight of the connected breathing circuit 3. The swivel coupling 2 enables the connected breathing circuit 3 to be displaced angularly in two planes orthogonal to one another and to the axis of the coupling. The swivel coupling 2 also allows relative rotational displacement of the two components 21 and 22 about the axis of the coupling so that the connected tubing 32 can rotate as it is moved without twisting and kinking. The compact construction of the coupling when fitted between the tracheostomy tube and the breathing circuit also helps minimise additional dead space.

The compact swivel coupling provided by the present arrangement can be a particular advantage in ambulatory applications where the patient is breathing spontaneously and is encouraged to walk around to aid recovery. The coupling can help reduce forces applied to the tracheostomy tube by the connected tubing, enabling the patient freer movement and comfort.

The coupling 2 can be fitted to conventional tracheostomy tubes without modification and can easily be removed to allow an inner cannula or other device to be inserted into and removed from the tracheostomy tube.

1-9. (canceled)

10. A coupling adapted for fitting on a male connector at the machine end of a tracheostomy tube, the coupling including a first component including a substantially cylindrical sleeve at one end having a female tapered bore adapted to form a tapered fit on the outside of the male connector, characterised in that the first component has at its opposite end an enlarged part-spherical formation having an open end aligned with the sleeve, that the coupling includes a second component having a machine end portion of substantially cylindrical shape with a male tapered external surface adapted to fit with a cooperating female tapered connector, that the patient end of the second component has a part-spherical formation with an open end, and that the part-spherical formation on the second component is located within the part-spherical formation on the first component and forms a close sliding fit between the outside of the formation on the second component and the inside of the formation on the first component such that the machine end of the second component can be displaced in two orthogonal planes relative to the first component.

11. A coupling according to claim 10, characterised in that the sleeve on the first component is shorter than the male connector on the tracheostomy tube and that the sleeve is arranged to fit towards the patient end of the male connector such that the machine end of the male connector extends into the part-spherical formation at the machine end of the first component.

12. A coupling according to claim 11, characterised in that the sleeve on the first component is arranged such that about half the length of the male connector extends in the part-spherical formation on the first component.

13. A coupling according to claim 10, characterised in that the coupling is arranged such that an end of the male connector projects in the open end of the part-spherical formation on the second component.

14. A tracheostomy tube assembly including a tracheostomy tube and a coupling fitted on a male connector at the machine end of the tracheostomy tube, the coupling including a first component including a substantially cylindrical sleeve at one end having a female tapered bore fitted on the outside of the male connector, characterised in that the first component has at its opposite end an enlarged part-spherical formation having an open end aligned with the sleeve, that the coupling includes a second component having a machine end portion of substantially cylindrical shape with a male tapered external surface adapted to fit with a cooperating female tapered connector, that the patient end of the second component has a part-spherical formation with an open end, and that the part spherical formation on the second component is located within the part-spherical formation on the first component and forms a close sliding fit between the outside of the formation on the second component and the inside of the formation on the first component such that the machine end of the second component can be displaced in two orthogonal planes relative to the first component.

15. A tracheostomy tube assembly according to claim 14, characterised in that the sleeve on the first component is shorter than the male connector on the tracheostomy tube, and that the sleeve is fitted towards the patient end of the male connector such that the machine end of the male connector extends into the part-spherical formation at the machine end of the first component.

16. A tracheostomy tube assembly according to claim 15, characterised in that the sleeve on the first component is arranged such that about half the length of the male connector extends in the part-spherical formation on the first component.

17. A tracheostomy tube assembly according to claim 14, characterised in that an end of the male connector projects in the open end of the part-spherical formation on the second component.

18. An airway system including a breathing circuit and a tracheostomy tube assembly, wherein the tracheostomy tube assembly includes a tracheostomy tube and a coupling fitted on a male connector at the machine end of the tracheostomy tube, the coupling including a first component including a substantially cylindrical sleeve at one end having a female tapered bore fitted on the outside of the male connector, the first component having at its opposite end an enlarged part-spherical formation having an open end aligned with the sleeve, the coupling including a second component having a machine end portion of substantially cylindrical shape with a male tapered external surface adapted to fit with a cooperating female tapered connector, the patient end of the second component having a part-spherical formation with an open end, and the part spherical formation on the second component being located within the part-spherical formation on the first component and forms a close sliding fit between the outside of the formation on the second component and the inside of the formation on the first component such that the machine end of the second component can be displaced in two orthogonal planes relative to the first component, and wherein the tracheostomy tube is connected with the breathing circuit via the coupling such that movement of the breathing circuit relative to the tracheostomy tube is accommodated by movement of the second component relative to the first component.
